(19) United States
(12) Patent Application Publication
(10) Pub. No.: US 2008/0195083 A1
(43) Pub. Date: Aug. 14, 2008

(54) Implantable bolus injector

(76) Inventors: Michael Axelsson, Landvetter (SE); Koullis Pitsillides, Sacramento, CA (US)

Correspondence Address: LYNN E BARBER, P O BOX 16528, FORT WORTH, TX, 76162, US

(21) Appl. No.: 12/011,525

(22) Filed: Jan. 28, 2008

Related U.S. Application Data
(60) Provisional application No. 60/898,503, filed on Jan. 31, 2007.

(51) Int. Cl. A61K 9/22

(52) U.S. Cl. 604/891.1

(57) ABSTRACT

An implantable bolus injector having a battery, an electronic control unit and an injector including a shape memory alloy spring, a standard low-tension spring, and a threaded top part with a stainless steel cannula. The electronic control unit is connected to the shape memory alloy spring via a direct connection to the lower part of the spring and via the low-tension spring that ensures a connection between the spring and the control unit during the expansion of the shape memory alloy spring.

### CROSS-REFERENCE TO RELATED APPLICATIONS

This application claims priority from U.S. provisional application Ser. No. 60/898,503 filed Jan. 31, 2007.

### BACKGROUND OF THE INVENTION

1. Field of the Invention

The present invention relates generally to biomedical devices, especially in the experimental biomedical field, where it makes it possible to continue pharmacological studies in animal models equipped with telemetric devices when no easy route for administration of various pharmacological substances is present. The device described in this invention is an implantable, telemetric, bolus injector of simple design with a shape memory material spring as an actuator.

2. Background

The use of telemetric techniques for recording of physiological variables from animals is steadily increasing both in the laboratory environment and in field experiments. This is a very positive development since it will lead to an increase in the quality of the recorded data due to a reduced post-surgical stress as well as a reduced confined induced stress. It also opens up new areas where traditional recording methods cannot be used. Telemetric techniques enable long term recordings without the risk of developing infection around skin penetrating catheters, flow probe cables or other exteriorized leads. They also make it possible to study animals in their natural environment (eco-physiology), something that is not possible with traditional recording methods. Historically the use of telemetry to transmit data between a subject and an investigator started in 1869 (1). This was followed by Einthoven's experiment in 1903 where he used a telephone line to transmit ECG data over a 1.5 km distance (2). With the modern definition of biotelemetry “measurements from unrestrained animal or patient using radiolinks” (3), the first scientist to use telemetric techniques was Winters who may be the inventor of telemetry in 1921 (4), followed by Fuller & Gordon in 1948 (5). The introduction of the transistor in 1952 was a breakthrough and smaller and better telemetric devices could be developed.

For experimental pharmacology, fully implanted telemetric equipment poses a problem, while the traditional recording methods with indwelling catheters allowed both recording of pressure, and sampling of fluid and injection of different pharmacologically active substances. Using fully implantable telemetric units, the only way to administer the drug is via the food or via direct injections, intramuscular, intraperitoneally or intravascular.

Administrating the drug via the food is difficult for many reasons, as some drugs may be degraded in the stomach and can therefore not be given orally, and it is also difficult to ensure a fixed dose at a specific time. Administrating drugs via injections induces stress in the animal since it has to be handled and this was the very reason for the use of the telemetric system to reduce postsurgical and experimental protocol induced stress.

One solution to this problem is to use a remotely controlled injector that can administer the drug at a given command. After a thorough search of the market and different patent databases it was obvious that this kind of bolus injector (injector delivering a prefixed volume in a short time) was not commercially available and no filed patents described such a device.

Shape memory alloys (SMAs) are materials that can return to a predetermined shape when heated. While a SMA is above its transformation temperature, it is capable of being formed into an original shape by certain metal-working techniques, among them, extrusion, forging, hot rolling, and forming. The SMA enters another state when cooled below its transformation temperature; in this state the SMA is capable of deformation. SMAs retain a deformed shape until heated above the transformation temperature, whereupon a change in crystal structure causes the SMA to return to its original shape. One attribute of SMAs is the ability to generate extremely large recovery stresses, i.e., exerting large forces, when constrained from returning to its original conformation. One example of a SMA is a nickel-titanium alloy called Nitinol.

Nitinol is a corrosion resistant, super elastic Nickel-Titanium alloy. Being super elastic, Nitinol can be stretched up to 10% without rupturing. When contracting, it can produce an actuation stress of more than 600 MegaPascal (MPa) in extreme cases, but should normally not be subjected to more than 170 MPa. Furthermore, when stretched less than 5%, Nitinol is also fatigue persistent. Required electric potential for power supply is normally a few volts to produce an actuation stress. Temperature characteristics for the phase change are highly dependent on the content ratio of the alloy components. There exist in addition to various types of Nitinol also, for example, Fe-based and Cu-based shape memory alloys.

U.S. Pat. No. 6,656,158 assigned to Insulet Corporation describes a pump mechanism that is driven by a straight shape memory metal wire. The length change in the wire during heating is transferred to via a cogwheel to a screw. The pump is designed for a precise delivery of small volume over long periods of time (infusion). The pump is complicated in its design with a number of moving parts. The size of the described invention is not stated and it is not clear if this device can be fully implanted which is a prerequisite in the invention herein.

The same applicants discloses a fluid delivery device in U.S. Pat. application 20050238507A1. This is a complex design for delivery of small volumes of fluid and also it uses straight shape memory alloy wire for the actuators. The change in the wire is transferred to a screw via a cogwheel mechanism. The screw is part of a piston in a syringe-like mechanism. This is not a bolus injector according to the specifications for the present invention. As in the abovementioned patent, it is not stated if this fluid delivery device is implantable, nor do the applicants mention the final size of the fluid delivery system.

The invention described in U.S. Pat. No. 6,375,638 (Medtronic MiniMed, Inc.) is based on the shape memory alloy and the fact that it changes length when heated. The invention uses a straight shape memory alloy wire and the length change in the wire is transferred to a piston or roller that is part of a syringe or reservoir system. Each heating cycle produces a small forward movement of the piston or roller. This invention produces small discrete steps and could not be considered to be a bolus injector. This is also not an implantable system and the size is too large to be useful in smaller research animals.

U.S. Pat. No. 6,743,204 discloses a rather sophisticated and complicated peristaltic pump using shape memory alloy springs to keep the rollers in the pump head against the tubing. The patent does not use the memory capacity of the shape memory alloy but instead use them as passive springs. This device cannot be considered to be a bolus injector.

U.S. Pat. application 20050192561 A1 assigned to M2 Medical, describes an infusion pump system which is yet another system for delivery of small volumes over extended periods of times. The invention uses straight shape memory wires as actuators. Several different ways of transferring the force from the shape memory alloy is described in the patent description (membrane pumps mechanisms or piston pump mechanism). Some drawings in this patent description can also neither of these patent applications describes a bolus injector and they are not suitable designs for implantation.

U.S. Pat. application US 20060013716 A1 discloses a shape memory alloy wire driven positive displacement micropump with pulsatile output. Mechanisms based on straight shape memory alloy wires are described in this patent application together with valve mechanisms for dispensing fluid. Two different types of pump mechanisms are described both based on shape memory alloy wires in combination with standard springs. Each stroke of the pump will deliver a small volume of fluid from a reservoir. Unlike the invention herein the device is not suitable for implantation and is not a telemetric device.

There are a number infusion-products on the market and several patents describing very complicated systems mainly for infusion of the drug over extended periods of time. Many of them seem to target insulin administration in diabetes patients where long term slow accurate infusion rates are important. However none of them relates to biomedical devices for studies in animal models equipped with telemetric devices.

The injector described in the invention herein uses a shape memory material (Nitinol) spring as an actuator, which is not the case in any of the listed patents where straight shape memory actuators have been used. All of the studied patents describe much more complicated systems mainly for infusion of the drug over extended periods of time. Many seem to be targeting insulin administration in diabetes patients where long term, slow and accurate infusion rates are important. Compared to these patents, the bolus injector herein has few parts and is relatively simple in its design. It is also different from the other described systems in that it delivers a prefixed volume in a short time (bolus dose). It also differs in that none of the other systems seems to be designed to be fully implantable in the subject.

Now, therefore the invention herein was made to address the above problems. It is an object of the invention herein to provide an injector ideal for remotely administering drugs to animal models, where no easy administrative route for various pharmacological substances is present. It is a further object of the invention to be implantable and of bolus injection type i.e. it should deliver a fixed volume over a short time period. Yet a further object of the invention is that the product has few components and has a simple design in order to be inexpensive to manufacture and reduce the risk of malfunction.

Other objects and advantages will be more fully apparent from the following disclosure and appended claims.

### SUMMARY

The present invention relates generally to biomedical devices, especially in the experimental biomedical field. The invention makes it possible to continue pharmacological studies in animal models equipped with telemetric devices where no easy administrative route for various pharmacological substances is present. The invention relates to an injector with few components in order to reduce the manufacturing cost of the injector with the goal of producing a single-use unit.

The invention herein is an implantable bolus injector having a battery, an electronic control unit and an injector including a shape memory alloy spring, a standard low-tension spring, and a threaded top part with a stainless steel cannula. The electronic control unit is connected to the shape memory alloy spring via a direct connection to the lower part of the spring and via the low-tension spring that ensures a connection between the spring and the control unit during the expansion of the shape memory alloy spring.

An object of the invention is to provide an injector of simple design with few moving parts to reduce the risk of malfunction. The invention is preferred to be fully implantable without any skin-penetrating parts. An other object of the invention is that the injector could be activated both via an external radio signal or via a direct connection to an telemetric implant. A further object of the invention is that it is of the bolus type.

### BRIEF DESCRIPTION OF THE DRAWINGS

FIG. 1 is a longitudinal cross-section of an exemplary embodiment of the invention showing a battery, an electronic control unit and the injector.

FIG. 2 is a longitudinal cross-section of an exemplary embodiment of the invention showing the internal components.

FIG. 3 illustrates embodiments of various parts of injector.

FIG. 4 shows the flow at the four tested output pressures.

FIG. 5 shows the lag time.

FIG. 6 shows the injection time.

### DETAILED DESCRIPTION OF THE INVENTION AND PREFERRED EMBODIMENTS THEREOF

The injector described in the invention herein is made with as few components as possible in order to reduce the cost of the injector with the goal of producing a single-use units. The design is simple, with few moving parts to reduce the risk of malfunction. Further the injector is fully implantable without any skin-penetrating parts. It may be activated either via a external radio signal or via a direct connection to an telemetric implant. The injector should be of bolus type, i.e., it should deliver a fixed volume (0.1-5 ml) over a short time period (less than 60 seconds). Further the injector should be able to inject against systemic vascular pressures up to 15 kPa. The preferred injector of the invention uses the shape memory material (Nitinol) spring as an actuator.

### EXAMPLE 1

### Actuator Design

The spring actuator can be ordered as a custom made part from any company working with Nitinol alloys or it can be made from a straight piece of Nitinol wire with only a few tools. The Nitinol alloy has a dynamic crystalline structure, with the molecular structure sensitive to stress and temperature. Martensite is the low temperature crystalline form and Austenite is the high temperature crystalline form. In the Martensite form, a straight piece of Nitinol wire can be shaped into a spring by winding it around a metal rod of suitable diameter. It is important to set the length of the spring at this point before heating it up to above its annealing temperature where the alloy will reorient its crystalline structure and “remember” its new form.

The Nitinol based spring actuator used in the present project was created using 0.77 mm (0.03″) diameter wire that was wound around a 3 mm metal rod to form an 18 mm long spring and then heated to above its annealing temperature. When cooled the spring can be compressed to 7 mm and stays that length until it is heated, when it again expands to 18 mm. The transition temperature and annealing temperatures vary depending on the exact composition of the alloy, but normally the transition temperature is found between 70 to 130° C. and the annealing temperature around 540° C.

### EXAMPLE 2

### Assembly of an Injector

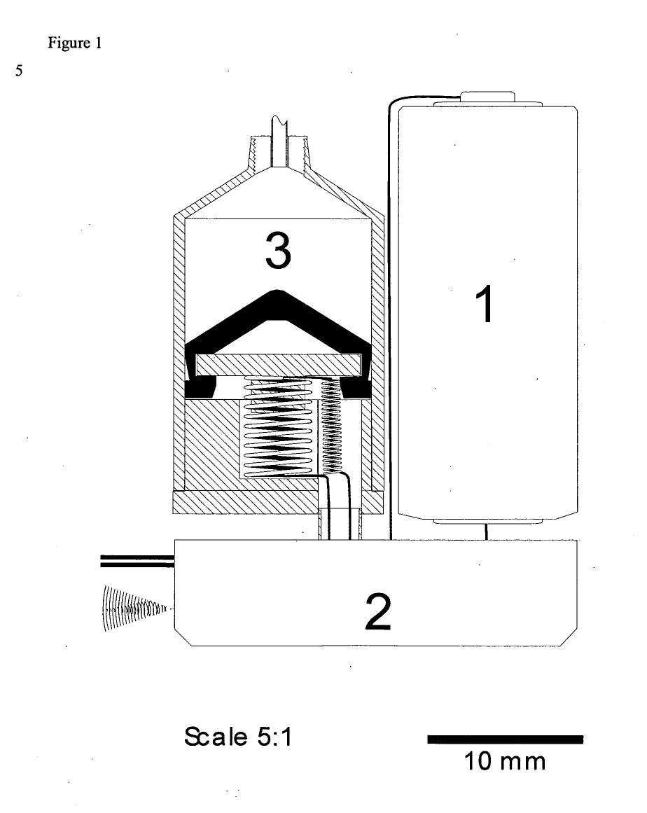

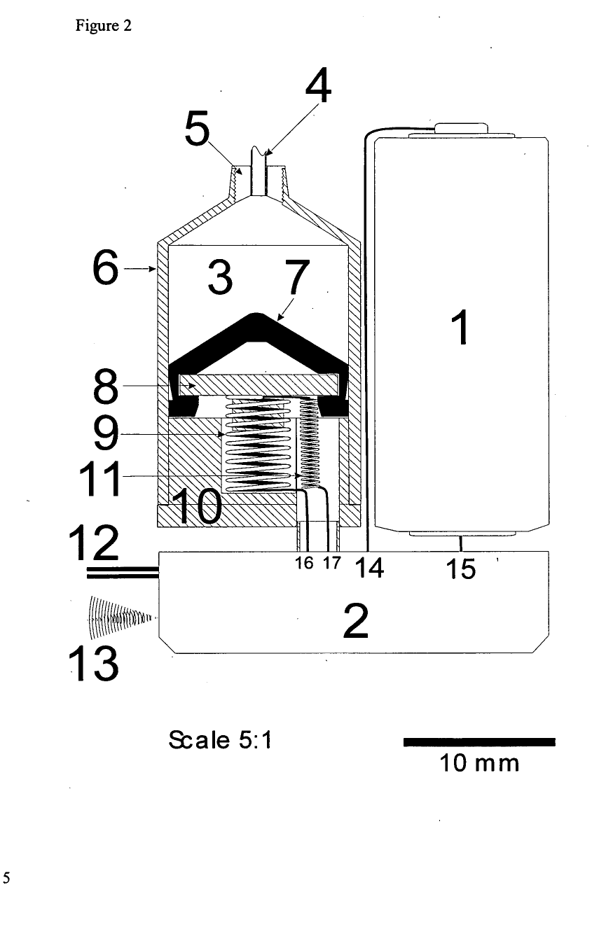

The injector in its assembled form is shown in FIGS. 1-2. It consists of three major parts, a battery (1), an electronic control unit (2) and the injector (3). In its final assembly, the entire injector is encased in silicon to ensure biocompatibility and protection again tissue fluids. The battery (1) is a standard commercially available alkaline high-energy 1.5V battery. The control unit (2) is a custom designed electronic control for the injector (3), and is the link between the battery (1) and the shape memory alloy spring (9), being controlled either via a direct hardwired (12) connection to an implanted telemetric device or via an external radio link (13).

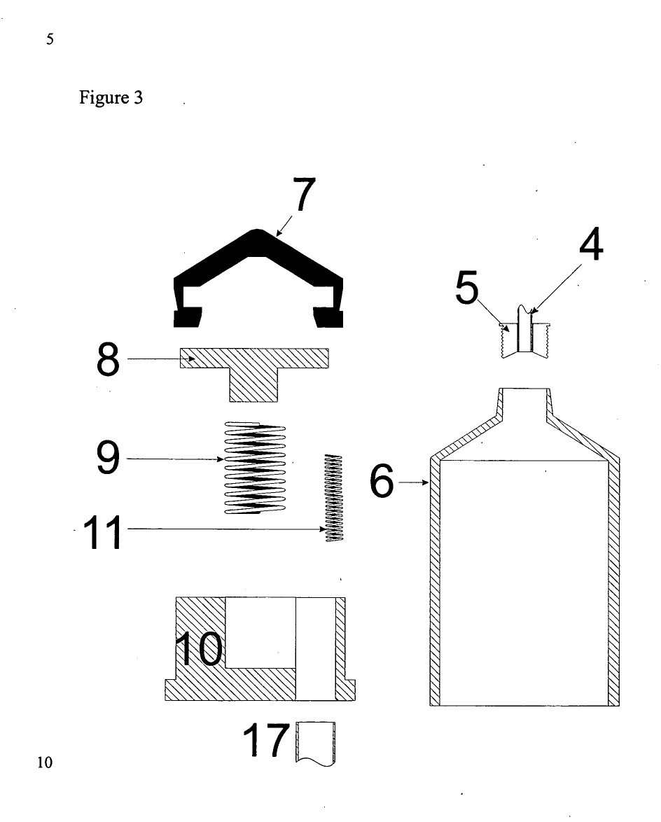

The injector (3) itself is shown in FIG. 3, it consists of a plastic housing (6) with an endplug (10), a rubber plunger (7) with a plastic support disk (8), the shape memory alloy spring (9), a standard low-tension spring (11), and a threaded top part (5) with a stainless steel cannula (4). The electronic control unit (2) is connected to the shape memory alloy spring (9) via a direct connection to the lower part of the spring (9) and via the low-tension spring (11) that ensures a connection between the spring (9) and the control unit (2) during the expansion of the shape memory alloy spring (9). The electronic control unit (2) is connected to the battery via two miniature connectors (14, 15).

The injector has to be loaded before implantation and cannot be reloaded once implanted so it can only be used for one injection per implantation. To load the injector the threaded top part (5) with its stainless steel cannula (4) is unscrewed and then the endplug (10) is lowered a few millimeters. The space above the rubber plunger (7) is filled with the pharmacological substance of choice using a syringe and a needle. The threaded top part (5) is then screwed back on and a catheter of choice (preferably a check valve type catheter) is attached to the stainless steel cannula (4). Holding the injector upright the endplug (10) is now advanced so that it sits tight against the lower part of the plastic housing (6) which ensures that the threaded top part (5), stainless steel cannula (4) and the catheter are filled with the pharmacological solution without any air bubbles in the system. After filling the system the entire injector is coated in medical grade silicon to ensure biocompatibility and protection of the electronics

### EXAMPLE 3

### Measuring the Injector Pressure and Flow Generation

In order to test and characterize the injector, the injector pressure and flow generation was measured at four different output pressures (0, 5, 10 and 15 kPa). At each output pressure, five consecutive tests were run. The results are presented as average+SD. To record pressure and flow generation, the injector was connected to a pressure transducer (model DPT-6100, pvb Medizintechnik, Kirchseeon, Germany) using a double bore cannula and an in-line flow-through transit time flow probe with an internal diameter of 1 mm (Transonic System Inc., Ithaca, N.Y., USA). The pressure transducer was calibrated against a static water column. The signals generated by the transducers were amplified using a 4ChAmp amplifier (Somedic, Hörby, Sweden). The flow probe was connected to a Transonic flow-meter (model T206; Transonic System Inc., Ithaca, N.Y., USA). Data were digitally stored for subsequent analysis using a Power Lab unit (ADInstruments Pty Ltd, Castle Hill, Australia) connected to a portable computer. The output pressure was set as a static water column.

Results

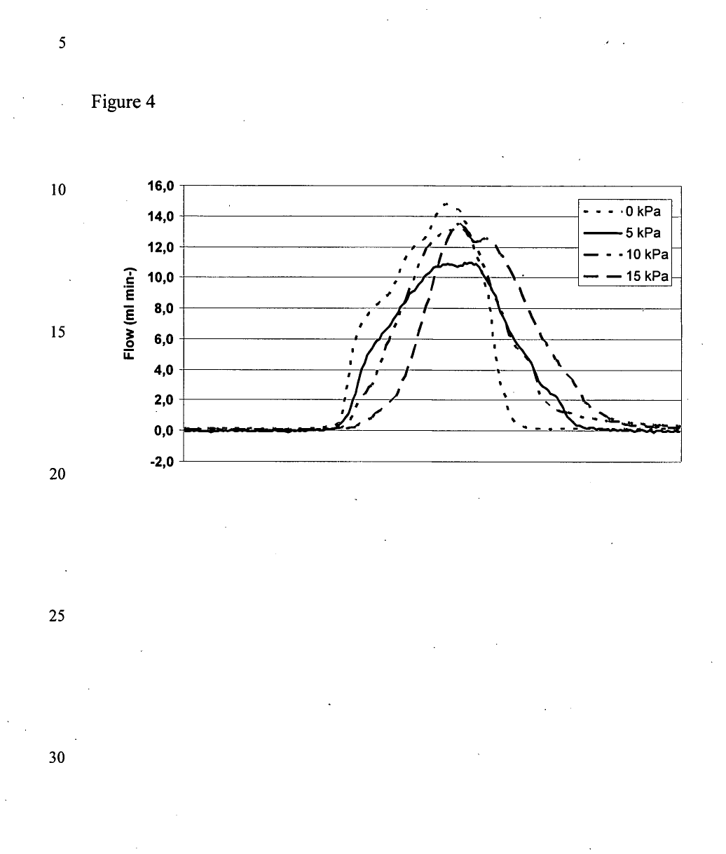

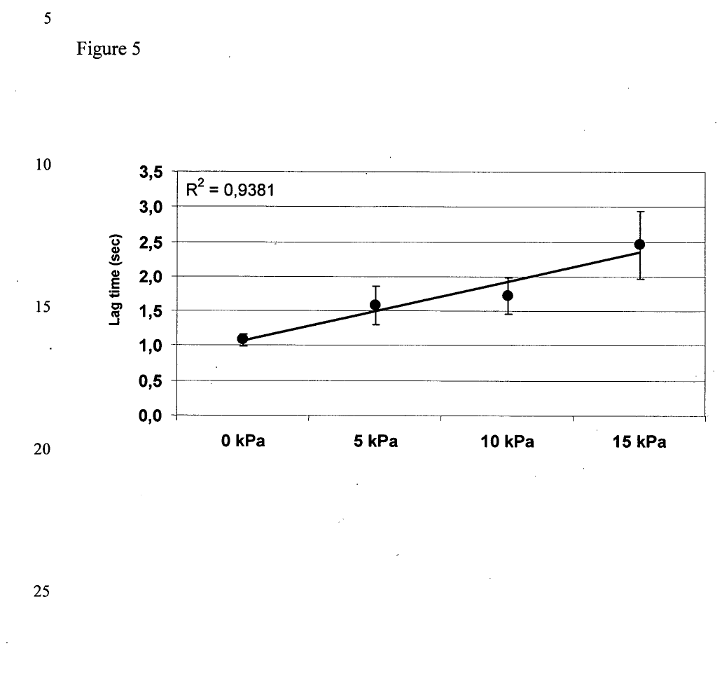

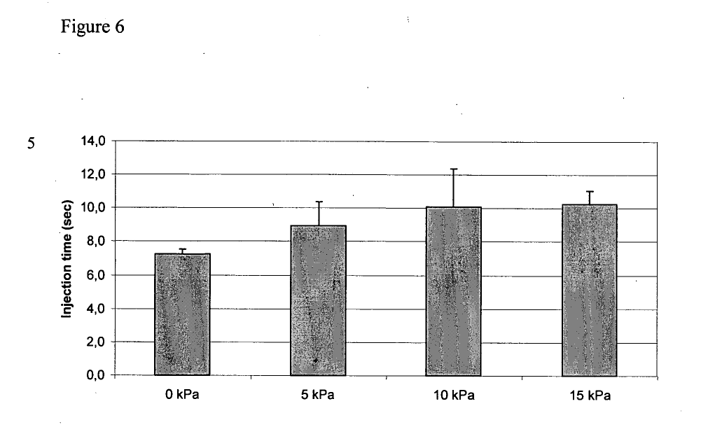

FIG. 4 shows flow at the four tested output pressures. At the highest tested output pressure (15 kPa), flow starts later, shows a longer lag time (see FIG. 5) and injection time is longer than at 0 kPa (see FIG. 6).

FIG. 5 shows the lag time. Lag time in this case was defined as the time until flow had increased above 0.5 ml min⁻. There is a strong correlation between lag time and output pressure and the difference between 0 and 15 kPa is 1.38+0.5 seconds.

In FIG. 6, injection time is plotted and in this case it is defined as the time between the lag time and the time when flow dropped below 0.5 ml min-1. There is an increase in injection time from 0 to 15 kPa with 3.2+0.8 seconds which corresponds to 44+12% increase.

In summary, the tests show that the criteria set up for the injector are met and even though it is affected by the output pressure, the injection time is well within the original specification of less than 60 seconds.

### REFERENCES

1. Marey M. Phenomenon of flight in the animal kingdom. In: Smithsonian Ann Rept Washington, D.C.: U.S. Gov. Print Office, 1896, 226-285.
2. Einthoven W. Galvanometrische registrierung des menschlichem elektrokardiograms. Arch ges Physiol 99: 472, 1903.
3. Cooke S J, Hinch S G, Wikelski M, Andrews R D, Kuchel L J, Wolcott T G, and Butler P J. Biotelemetry: a mechanistic approach to ecology. Trends in Ecology and Evolution 19: 334-343, 2004.
4. Winters S R. Diagnostics by wireless. Sci Am 124: 465, 1921
5. Fuller S L and Gordon T M. The radio inductograph, a device for recording physiological activity in unrestrained animals. Science 108: 287, 1948.

1. An implantable bolus injector device comprising a battery, an electronic control unit and an injector comprising a shape memory alloy spring as an actuator and a low-tension spring, wherein the electronic control unit is connected to the shape memory alloy spring via the low-tension spring, wherein the injector device has no skin-penetrating parts.

2. The implantable bolus injector device of claim 1, wherein the injector is activated remotely via an external radio signal.

3. The implantable bolus injector device of claim 1, wherein the injector is activated via a direct connection to a telemetric implant.

4. The implantable bolus injector device of claim 1, wherein the injector delivers a fixed volume over a particular time period.

5. The implantable bolus injector device of claim 4, wherein the fixed volume is 0.1 to 5 ml and the particular time period is less than 60 seconds.

6. The implantable bolus injector device of claim 1, wherein the injector can inject against vascular pressures up to 15 kPa.

7. The implantable bolus injector device of claim 1, wherein the shape memory alloy spring is made of Nitinol.

8. The implantable bolus injector device of claim 1, wherein the shape memory alloy spring is made of a wire wound around a metal rod of suitable diameter, and then the length of the spring is set before heating the spring above its annealing temperature.

9. The implantable bolus injector of claim 8, wherein the wire has a diameter of 0.77 mm and was formed by being wound around a 3 mm metal rod to form an 18 mm long spring.

10. The implantable bolus injector device of claim 1, wherein the shape memory alloy spring is made of a material selected from the group consisting of nickel, iron and copper-based shape memory alloys.

11. The implantable bolus injector device of claim 1, wherein the injector device is encased in silicon.

12. The implantable bolus injector device of claim 1, wherein the control unit links between the battery and the shape memory alloy spring.
